(19) United States
(12) Patent Application Publication
(10) Pub. No.: US 2004/0077591 A1
(43) Pub. Date: Apr. 22, 2004

(54) Histone deacetylase inhibitors for the treatment of multiple sclerosis, amyotrophic lateral sclerosis and Alzheimer's Disease

(75) Inventor: Fernando Dangond, Newton, MA (US)

Correspondence Address: FULBRIGHT & JAWORSKI L.L.P., SUITE 2400, 600 CONGRESS AVENUE, AUSTIN, TX, 78701-3271, US

(73) Assignee: The Brigham and Women's Hospital, Inc.

(21) Appl. No.: 10/401,274

(22) Filed: Mar. 27, 2003

Related U.S. Application Data
(60) Provisional application No. 60/368,228, filed on Mar. 28, 2002.
(60) Provisional application No. 60/404,664, filed on Aug. 20, 2002.

(51) Int. Cl. A61K 31/739; A61K 31/40; A61K 31/19; A61K 31/192

(52) U.S. Cl. 514/54; 514/557; 514/570; 514/408; 514/575

(57) ABSTRACT

The present invention provide therapies for Alzheimer's Disease (AD), multiple sclerosis (MS) and amyotrophic lateral sclerosis (ALS). The method relies on the use of an HDAC inhibitor, alone or in combination with other drugs, to prevent or treat AD, MS or ALS. Also provided are methods of screening for additional HDAC inhibitors with particular efficacy against these disease states.

This application claims benefit of priority U.S. Provisional Application Serial No. 60/368,228, filed Mar. 28, 2002, and 60/404,664, filed Aug. 20, 2002, the entire contents of both hereby being incorporated by reference.

This invention was made with Government support under NIH Grant No. 5K08CA080084 awarded by the PHS. The Government has certain rights in the invention.

### BACKGROUND OF THE INVENTION

I. Field of the Invention

The present invention relates generally to the fields of neuropathology and molecular biology. More particularly, it concerns the use of inhibitors of histone deacetylases to treat specific neuropathologies, namely, Alzheimer's Disease, multiple sclerosis and amyotrophic lateral sclerosis.

II. Description of Related Art

Neurodegenerative diseases are generally characterized by the loss of neurons from one or more regions of the central nervous system. They are complex in both origin and progression, and have proved to be some of the most difficult types of disease to treat. In fact, for some neurodegenerative diseases, there are no drugs available that provide significant therapeutic benefit. The difficulty in providing therapy is all the more tragic given the devastating effects these diseases have on their victims.

A. Multiple Sclerosis

MS is an inflammatory, demyelinating disease of the human brain and spinal cord. MS lesions are characterized by perivenular infiltration of activated monocytes and lymphocytes. MS lesions appear as multifocal, often confluent areas of demyelination and are associated with variable degrees of oligodendrocyte and axonal loss and gliosis, on a background of edema. The immune system activation in MS is thought to be responsible for eventually triggering neurodegeneration.

MS can affect practically any age group but it is most commonly diagnosed in individuals between the ages of 18-50 years. Clinical exacerbations in MS are neurological deficits which typically last more than a day, usually several days. MS presents in different forms, such as relapsing remitting (70%), primary progressive (15%), and relapsing progressive (15%). Nearly two thirds of patients with relapsing remitting disease eventually develop a progressive type of MS, known as the secondary progressive form.

B. Amyotrophic Lateral Sclerosis

Amyotrophic Lateral Sclerosis (ALS) is a fatal, age-dependent human neurodegenerative disease of the central nervous system (CNS), characterized by loss of motor neurons in the brain, brainstem and spinal cord. Familial and sporadic ALS are pathologically and clinically similar, leading to death, on average, in approximately 5 years. Approximately 90% of ALS cases occur sporadically. Of the familial ALS cases, only a small fraction (<15%) has been found to be associated with dominant mutations in the cytosolic, Cu/Zn superoxide dismutase 1 (SOD1) gene, or in a Rho GTPase-like gene, named Alsin. SOD1 catalyzes the dismutation of the toxic superoxide anion O₂⁻ to molecular O₂ and H₂O₂. An autosomal dominant form of juvenile ALS has been mapped to 9q34. In addition, other pedigrees with non-SOD1 dominant ALS forms have been described. However, the defective genes within these loci have not yet been identified. Although the disease subtypes may have multiple etiologies, eventual loss of motor neurons may be the result of commonly shared downstream molecular pathways whose regulation becomes altered. A common phenotype, for instance, could be an impairment of hydrogen peroxide detoxification pathways, resulting in elevated oxidation of DNA, protein and membrane phospholipids, which primarily affects motor neurons.

C. Alzheimer's Disease

Dementia is a brain disorder that seriously affects a person's ability to carry out daily activities. Alzheimer's disease (AD) is the most common form of dementia among older people. Scientists believe that up to 4 million Americans suffer from AD. The disease usually begins after age 60, and risk goes up with age. While younger people also may get AD, it is much less common. About 3 percent of men and women ages 65 to 74 have AD, and nearly half of those age 85 and older may have the disease. While the subject of intensive research, the precise causes of AD are still unknown, and there is no cure.

AD attacks parts of the brain that control thought, memory, and language. It was named after Dr. Alois Alzheimer, a German doctor. In 1906, Dr. Alzheimer noticed changes in the brain tissue of a woman who had died of an unusual mental illness. He found abnormal clumps (now called amyloid plaques) and tangled bundles of fibers (now called neurofibrillary tangles). Today, these plaques and tangles in the brain are considered hallmarks of AD.

Scientists also have found other brain changes in people with AD. There is a loss of nerve cells in areas of the brain that are vital to memory and other mental abilities. There also are lower levels of chemicals in the brain that carry complex messages back and forth between nerve cells. Thus, AD may disrupt normal thinking and memory by inhibiting, both physically and chemically, the transfer of message between nerve cells.

D. HDAC Inhibitors

Chang et al. (2001) examined the effects of HDAC inhibitors on spinal muscular atrophy (SMA). This study showed that sodium butyrate was effective at increasing the amount of exon 7-containing survival motor neuron (SMN) protein in SMA lymphoid cell lines by changing the alternative splice pattern of exon 7 in the SMN2 gene, which has been shown to be protective from SMA effects. In vivo, SMA-like mice showed increased expression of SMN protein in spinal cord motor neurons when treated with sodium butyrate, as well as improved symptoms. The drug also decreased the birth rate of severe types of SMA-like mice when heterozygous knock-out transgenic SMA-like mice were bred.

Steffan et al (2001) explored the ability of HDAC inhibitors to affect Huntington's Disease. There, it was shown that HDAC inhibitors could reverse the reduction in acetylated H3 and H4 histones caused by Httex1p in cell free assays, a protein that binds the acetyltransferase domains of CREB-binding protein and p300. In vivo, HDAC inhibitors arrest ongoing progressive neuronal degeneration induced by polyglutamine repeat expansion, and reduce lethality in two Drosophila models of polyglutamine disease, suggesting a potential role in treatment of Huntington's Disease and other polyglutamine-repeat diseases.

### SUMMARY OF THE INVENTION

Thus, in accordance with the present invention, there is provided a method for treating or preventing amyotrophic lateral sclerosis (ALS) in a human subject comprising administering to the subject a therapeutic amount of a histone deacetylase (HDAC) inhibitor. The HDAC inhibitor may be selected from the group consisting of trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1 H-pyrrol-2-yl)-N-hydroxy-2-propenamide, suberoylanilide hydroxamic acid, FK228 and m-carboxycinnamic acid bis-hydroxamide.

Treating may comprise reducing one or more symptoms of ALS, such as focal or generalized motor weakness including progressive inability to walk or use limbs, spasticity, respiratory insufficiency, inability to swallow, choking, weight loss, muscle atrophy, muscle fasciculations, increased reflexes, progressive inability to perform activities of daily living, and/or shortened life span. Treating may also comprise inhibiting the progression of ALS. Preventing ALS may comprise identifying a subject at risk of ALS. The HDAC inhibitor may be administered orally, intraperitoneally, intrathecally, intravenously, intranasally, intraparenchymally, subcutaneously, intramuscularly, intravenously, dermally, or intrarectally.

The invention may further comprise administering a second agent in addition to the HDAC inhibitor. The second agent may be a second HDAC inhibitor or Riluzole. The second agent may be provided before the HDAC inhibitor, after the HDAC inhibitor, or provided at the same time as the HDAC inhibitor. The second HDAC inhibitor may be provided through the same or a different route than the first HDAC inhibitor, such as orally, intraperitoneally, intrathecally, intravenously, intranasally, intraparenchymally, subcutaneously, intramuscularly, intravenously, dermally, or intrarectally.

In another embodiment, there is provided a method for treating or preventing multiple sclerosis (MS) in a human subject comprising administering to the subject a therapeutic amount of a histone deacetylase (HDAC) inhibitor. The HDAC inhibitor may be selected from the group consisting of trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228 and m-carboxycinnamic acid bis-hydroxamide.

Treating may comprise reducing one or more symptoms of MS, such as dementia symptoms, decreased concentration, memory loss, inappropriate social affect, bipolar disorder symptoms, social disinhibition, decreased visuospatial abilities, blindness, decreased vision, decreased visual depth perception, decreased gaze fixation, ocular pain, abnormal eye movements, facial pain, abnormal facial movements, tinnitus, hoarse speech, choking, urinary incontinence, urgency, hesitancy, or retention, fecal incontinence, constipation, or obstipation, muscular weakness, limb spasms/cramps, inability to walk or grab objects due to weakness and incoordination, muscle atrophy, stiffness, impotence, loss of libido, vaginal pain or numbness sensation, pelvic spasms, anorgasmia, tingling, numbness, abnormal sensory perception, intolerance to heat, focal or generalized pain, sciatica pain, reflex sympathetic dystrophy, inability to perceive vibration or position changes, electric shock-like sensation going down the spine or limbs following flexion of the neck, fatigue, tiredness, head titubation, tremors, loss of balance, slurred speech, vertigo, or recurrent clinical deterioration. Treating may also comprise inhibiting the progression of MS. Preventing MS may comprise identifying at subject at risk of MS. The HDAC inhibitor may be administered orally, intraperitoneally, intrathecally, intravenously, intranasally, intraparenchymally, subcutaneously, intramuscularly, intravenously, dermally, and intrarectally.

The method may further comprise administering a second agent in addition to the HDAC inhibitor. The second agent may be a second HDAC inhibitor, or selected from the group consisting of methylprednisolone, prednisolone, interferon-β1a, interferon-β1b, glatiramer acetate, and mitoxantrone. The second agent may be provided before the HDAC inhibitor, after the HDAC inhibitor, or provided at the same time as the HDAC inhibitor. The second HDAC inhibitor may be provided through the same or a different route than the first HDAC inhibitor, such as orally, intraperitoneally, intrathecally, intravenously, intranasally, intraparenchymally, subcutaneously, intramuscularly, intravenously, dermally, or intrarectally.

The method may further comprise comparing the one or more symptoms to a comparable animal when treated with the HDAC inhibitor or the second agent alone. For example, the second agent is provided before the HDAC inhibitor, provided after the HDAC inhibitor, or provided at the same time as the HDAC inhibitor.

An additional embodiment comprises a pharmaceutical composition comprising an HDAC inhibitor and a drug useful for treating amyotrophic lateral sclerosis and/or multiple sclerosis. The HDAC inhibitor may be from trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228 and m-carboxycinnamic acid bis-hydroxamide. The drug may be methylprednisolone, prednisolone, interferon-β1a, interferon-β1b, glatiramer acetate, and mitoxantrone for MS, or Riluzole for ALS.

Further, there is provided a method for treating or preventing Alzheimer's Disease (AD) in a human subject comprising administering to the subject a therapeutic amount of a histone deacetylase (HDAC) inhibitor. The HDAC inhibitor may be selected from the group consisting of trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228 and m-carboxycinnamic acid bis-hydroxamide.

Treating may comprise reducing one or more symptoms of AD, such as progressive memory loss (usually first and foremost), emotional disturbance, anxiety, affect reduction, spatial disorientation, decreased attention, lack of motor skill initiation, difficulty naming, problems calculating or keeping track of recent events, loss of interest in daily or social activities, and inappropriate judgment. In addition, symptoms at the late stages such as incoherent speech content (aphasia), mutism, delirium, paranoia, myoclonic (jerky) movements and urinary incontinence may be prevented by treatment with HDAC inhibitors. Treating may also comprise inhibiting the progression of AD. Preventing AD may comprise identifying a subject at risk of AD. The HDAC inhibitor may be administered orally, intraperitoneally, intrathecally, intravenously, intranasally, intraparenchymally, subcutaneously, intramuscularly, intravenously, dermally, or intrarectally.

The invention may further comprise administering a second agent in addition to the HDAC inhibitor. The second agent may be a second HDAC inhibitor, or FDA-approved drugs for AD, such as tacrine (Cognex), donepezil (Aricept), rivastigmine (Exelon), and galantamine (Reminyl). The second agent may also be a nonsteroidal anti-inflammatory agent (NSAID), vitamin E, stimulant medications such as Methylphenidate (Ritalin or Concerta), Sibutramine (Meridia), or Modafinil (Provigil) (used to enhance concentration ability or diurnal wakefulness), and natural products such as gingko biloba or huperzine A (from the club moss Huperzia serrata) and their extracts. The second agent may be provided before the HDAC inhibitor, after the HDAC inhibitor, or provided at the same time as the HDAC inhibitor. The second HDAC inhibitor may be provided through the same or a different route than the first HDAC inhibitor, such as orally, intraperitoneally, intrathecally, intravenously, intranasally, intraparenchymally, subcutaneously, intramuscularly, intravenously, dermally, or intrarectally.

An additional embodiment comprises a pharmaceutical composition comprising an HDAC inhibitor and a drug useful for treating AD. The HDAC inhibitor may be selected from trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228 and m-carboxycinnamic acid bis-hydroxamide. The drug may be selected from any of the FDA-approved drugs for AD such as tacrine (Cognex), donepezil (Aricept), rivastigmine (Exelon), or galantamine (Reminyl). Said second agent may also be a nonsteroidal anti-inflammatory agent (NSAID), vitamin E, stimulant medications such as Methylphenidate (Ritalin or Concerta), Sibutramine (Meridia), or Modafinil (Provigil) (used to enhance concentration ability or diurnal wakefulness), and natural products such as Gingko biloba or huperzine A and their extracts.

In still yet another embodiment, there is provided a method of screening a histone deacetylase (HDAC) inhibitor for use in treating or preventing Alzheimer's Disease (AD), amyotrophic lateral sclerosis (ALS) or multiple sclerosis (MS) comprising (a) providing a suitable animal model for AD, ALS or MS; (b) administering at least a first HDAC inhibitor to the animal; and (c) assessing one or more symptoms of AD, ALS or MS on the animal, wherein an improvement in the one or more symptoms, as compared to a comparable animal not treated with the HDAC inhibitor, indicates that the HDAC inhibitor is useful in treating or preventing AD, ALS or MS. The method may screen for ALS therapy using the SOD1 G93A mutant mouse model, may screen for MS therapy using the experimental autoimmune encephalomyelitis (EAE) model, or may screen for AD therapy using Alzheimer's disease mouse models. Symptoms comprise inability to perform water maze tests, inability to associate unpleasant stimuli with an outcome (altered conditioning response), decreased locomotor activity, and decreased grip strength (AD), and weakness, tremors, paralysis, spasticity, incontinence, abnormal behavior (ALS and MS), and ataxia or blindness (MS). The screening may comprise administering to the animal a second agent, such as a second HDAC inhibitor or is selected from the group consisting of methylprednisolone, prednisolone, interferon-β1a, interferon-β1b, glatiramer acetate, and mitoxantrone for MS, Riluzole for ALS, or tacrine (Cognex), donepezil (Aricept), rivastigmine (Exelon), galantamine (Reminyl), an NSAID, vitamin E or Gingko biloba or its extracts for AD.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Efficacy of HDAC inhibitor treatment in reducing the clinical manifestations of experimental autoimmune encephalomyelitis (EAE) in mice. Mean clinical scores reflect reduced clinical disability by TSA in the neurodegenerative phase of EAE (● TSA-treated; ◯ vehicle-treated) (*P<0.05 by Fisher's protected least significant difference (PLSD) test). The remission phase lasted from days 19 to 34 following disease induction in a first experiment, days 21 to 32 in a second experiment. Animals were scored for signs of disability using the following widely used scale: 1, limp tail or isolated weakness of gait without limp tail; 2, partial hind leg paralysis; 3, total hind leg or partial hind and front leg paralysis and/or urinary incontinence; 4, total hind leg and partial front leg paralysis; 5, moribund or dead animal (with minor modifications from Issazadeh et al. (1998)). The definitions used were: (1) mean peak remission: mean of maximal score reached during remission, and (2) mean % days of severity: mean % of days with scores ≧2.

FIGS. 2A-2H—SOD1 G93A mutant ALS animals on no treatment on day 125.

FIGS. 3A-3F—G93A mutant ALS mouse on oral sodium phenylbutyrate on day 125. 1 mg/ml in drinking water, equivalent to a calculated dose of 1 mg/mg of body weight/day), shown on day 125.

FIG. 4—Comparison of treated and untreated SOD1 G93A ALS mice on day 125.

FIGS. 5A-5K—Phenylbutyrate-treated SOD1-mutant ALS animal at day 138.

FIG. 6—Treatment of ALS SOD1G93A mice with oral sodium phenylbutyrate (SPB). Dose of 1 mg SPB/ml of drinking water, started on day 64 of age. Scoring (adapted for ALS) is as follows: 1, limp tail or hind limb weakness, righting reflex <5 sec; 1.5, limp tail or hind limb weakness, righting reflex >5 sec; 2, limp tail and hind limb weakness; 2.5, partial hind limb paralysis; 3, total hind limb paralysis; 3.5, complete hind limb paralysis and partial front limb paralysis; 4, complete paralysis.

FIGS. 7A-B—Densitometry of bands from western analysis of brain tissue proteins isolated from HDAC inhibitor-treated and vehicle-treated mice. Antibodies recognizing the inactive and active forms of both caspase 3 (BD Biosciences, CA) and caspase 9 (Stressgen, CA) were used. There is a higher ratio of inactive caspase 3 (Pro-caspase 3) to active caspase 3 in the HDAC-inhibitor-treated mice. Activated caspase 9 is decreased by oral sodium phenylbutyrate (SPB) and by intraperitoneal TSA. Both findings correlate with clinical improvement in these animal models.

FIG. 8—Identification of gene expression changes induced by HDAC inhibitors in vivo, using reverse transcriptase polymerase chain reaction (RT-PCR). CNS and spleen tissue samples from EAE mice were used to isolate RNA. One step real time quantitative RT-PCR (QRT-PCR) was then performed in triplicate for each sample (n=3 per group of mice) with SYBR Green and an ABI Prism 7700 Sequence Detection System (PE Applied Biosystems, Foster City, Calif.). Bar graph and standard error of mean (SEM) representation shows key genes altered by TSA in EAE mice with >1.5 fold change as compared to vehicle-treated EAE animals.

### DESCRIPTION OF ILLUSTRATIVE EMBODIMENTS

Neurodegenerative diseases are attracting more and more attention. The diseases are particularly devastating in that they progressively incapacitate their victims, leading to billions of dollars in health care costs each year. Remarkably, though much progress has been made in recent years, there remain relatively few drugs that are useful in the treatment of neurodegenerative diseases, and almost none that are effective for a high percentage of patients. Thus, there is an urgent need for new and improved drugs and methods of therapy for these conditions, which include Alzheimer's Disease, multiple sclerosis and amyotrophic lateral sclerosis, the latter better known as “Lou Gehrig's Disease.”

I. The Present Invention

As discussed above, HDACs play a key role in the regulation of gene expression. Their participation in cancer development has been explored extensively, and their role in immune function is now being more clearly elucidated. As a result, a number of inhibitors of HDAC function have now been developed and are being tested for efficacy in various disease models.

In neuropathologies, the understanding of HDAC function is less clear. Two groups have explored the use of HDAC inhibitors in the treatment of neurodegenerative disease—Huntington's Disease and spinal muscular atrophy—and shown amelioration of disease in animal models. The present invention extends this work by exploring the effect of HDAC inhibitor therapy on three distinct neurodegenerative diseases Alzheimer's Disease (AD), amyotrophic lateral sclerosis (ALS) and multiple sclerosis (MS). The present inventor has demonstrated that histone deacetylase inhibitors ameliorate physical disability in animal models of MS and ALS. In the MOG-induced EAE mouse model, animals reached the peak of disability typically seen after immunization, but their physical disability was reduced significantly following the immune attack, suggesting an indirect or direct neuroprotective effect of HDAC inhibitor treatment. In the G93A SOD 1-mutant ALS mice, treatment with HDAC inhibitors led to significant delay in the appearance of first symptoms, more preservation of body weight, reduced atrophy and weakness, and extended survival. Thus, the use of HDAC inhibitors as neuropreventative and neurotherapeutic agents is disclosed. These and other aspects of the invention are described in greater detail below.

II. Neurodegenerative Diseases

There are a number of diseases that involve the degeneration of nervous system tissue. These pathologies are together considered “neurodegenerative” diseases, and can include such diverse maladies as Alzheimer's Disease, amyotrophic lateral sclerosis (ALS), corticobasal degeneration, Creutzfeldt-Jakob Disease, dementia with Lewy Bodies, frontal lobe degeneration, Huntington's Disease, Lewy Body variant Alzheimer's Disease, multiple sclerosis (MS), multiple system atrophy, multi-infarct dementia, neuronal intranuclear inclusion disease, Parkinson's Disease, Pick's Disease, prion-related diseases, progressive supranuclear palsy, tauopathies, and tri-nucleotide repeat diseases. Of these, the present invention deals particularly with multiple sclerosis (MS), amyotrophic lateral sclerosis (ALS) and Alzheimer's Disease (AD).

A. Multiple Sclerosis

Multiple sclerosis is one of the most common diseases of the nervous system, afflicting people of virtually all ages around the world, although it has a special preference for young people, especially women, and for those who grew up in northern latitudes. It has become increasingly clear that MS is not only characterized by central nervous system inflammation, but also by oxidative and cytotoxic stress, and neuronal and axonal damage, leading to brain and spinal cord atrophy and clinical disability, all representing typical aspects of a neurodegenerative disease. MS likely involves genetic susceptibility, but it does not appear to be directly inherited by a typical mendelian pattern. It usually causes sudden neurologic symptoms including vision loss, paralysis, numbness, and walking difficulties. The symptoms can be diverse and confusing, often coming and going without any pattern, making it difficult to diagnose, even today.

The symptoms are the result of changes in brain and spinal cord nerves, which lose their ability to transmit signals. Myelin, a complex substance that surrounds nerve fibers, is crucial for electrical conduction. In MS, myelin is destroyed by cells and proteins of the body's immune system, which normally defend the body against infections. The specific mechanism which triggers the self-destructive immune onslaught is unknown, although a viral infection is among the leading candidates.

In 1935, researchers demonstrated that self-reactivity to nerve tissue underlined the MS-like illness. Injecting myelin into laboratory animals under the proper conditions could induce an immune attack against the animals' own myelin, producing a disease very similar to MS. This laboratory animal form of MS, called experimental allergic (or autoimmune) encephalomyelitis, or EAE, has become an important model for studying the immunology and treatment of MS. Additional studies of EAE showed that EAE could be transmitted by transferring T cells from an affected animal to a well one, demonstrating the autoimmune nature of the disease.

Starting in 1969, the first successful scientific clinical trial for MS was conducted. Patients having acute attacks of MS were given the steroid ACTH, which proved superior in speeding recovery. This primitive intramuscular steroid therapy would give way to the modern steroid therapy still in use today for acute exacerbations. It also provided a lead in to later studies, such as those using interferons, substances that modulate the immune system. The first studies of β-interferon for MS began at the end of the 1970's.

In 1993, Betaseron® was approved by the FDA to reduce the severity and frequency of attacks. In 1996, Avonex® was approved to slow the development of disability and reduce the severity and frequency of attacks. A third drug, proven to affect the natural course of MS, Copaxone® (known generically as glatiramer acetate for injection but not an interferon), was launched in 1997. Recently, mitoxantrone (Novantrone®, a chemotherapeutic agent) and Rebif® (an interferon drug) have been approved by the FDA and added to the armamentarium to treat MS. Other therapies are under investigation, including intravenous immunoglobulins. Other possible therapies include remyelination and repair of nerve damage.

B. Amyotrophic Lateral Sclerosis

Amyotrophic lateral sclerosis (ALS), sometimes called Lou Gehrig's Disease, affects as many as 20,000 Americans at any given time, with 5,000 new cases being diagnosed in the United States each year. ALS affects people of all races and ethnic backgrounds. Men are about 1.5 times more likely than women to be diagnosed with the disease. ALS strikes in the prime of life, with people most commonly diagnosed between the ages of 40 and 70. However, it is possible for individuals to be diagnosed at younger and older ages. About 90-95% of ALS cases occur at random, meaning that individuals do not have a family history of the disease and other family members are not at increased risk for contracting the disease. In about 5-10% of ALS cases there is a family history of the disease.

ALS is a progressive neurological disease that attacks neurons that control voluntary muscles. Motor neurons, which are lost in ALS, are specialized nerve cells located in the brain, brainstem, and spinal cord. These neurons serve as connections from the nervous system to the muscles in the body, and their function is necessary for normal muscle movement. ALS causes motor neurons in both the brain and spinal cord to degenerate, and thus lose the ability to initiate and send messages to the muscles in the body. When the muscles become unable to function, they gradually atrophy and twitch. ALS can begin with very subtle symptoms such as weakness in affected muscles. Where this weakness first appears differs for different people, but the weakness and atrophy spread to other parts of the body as the disease progresses.

Initial symptoms may affect only one leg or arm, causing awkwardness and stumbling when walking or running. Subjects also may suffer difficulty lifting objects or with tasks that require manual dexterity. Eventually, the individual will not be able to stand or walk or use hands and arms to perform activities of daily living. In later stages of the disease, when the muscles in the diaphragm and chest wall become too weak, patients require a ventilator to breathe. Most people with ALS die from respiratory failure, usually 3 to 5 years after being diagnosed; however, some people survive 10 or more years after diagnosis.

Perhaps the most tragic irony of ALS is that it does not impair a person's mind, as the disease affects only the motor neurons. Personality, intelligence, memory, and self-awareness are not affected, nor are the senses of sight, smell, touch, hearing, and taste. Yet at the same time, ALS causes dramatic defects in an individual's ability to speak loudly and clearly, and eventually, completely prevents speaking and vocalizing. Early speech-related symptoms include nasal speech quality, difficulty pronouncing words, and difficulty with conversation. As muscles for breathing weaken, it becomes difficult for patients to speak loud enough to be understood and, eventually, extensive muscle atrophy eliminates the ability to speak altogether. Patients also experience difficulty chewing and swallowing, which increase over time to the point that a feeding tube is required.

C. Alzheimer's Disease

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation.

Presenilin encodes the protease responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory.

Similarly, ApoE, which is involved in the redistribution of cholesterol, is now believed to contribute to AD development. Individuals having the ε4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

III. Histone Deacetylases and Inhibitors Thereof

Nucleosomes, the primary scaffold of chromatin folding, are dynamic macromolecular structures, influencing chromatin solution conformations (Workman and Kingston, 1998). The nucleosome core is made up of histone proteins, H2A, H2B, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

More than twelve different HDACs have been cloned from vertebrate organisms. The first three human HDACs identified were HDAC 1 (Taunton et al., 1996), HDAC 2 (Yang et al., 1996) and HDAC 3 (Dangond et al., 1998; Yang et al., 1997; Emiliani et al., 1998) (termed class I human HDACs). Recently class II human HDACs, HDAC 4, HDAC 5, HDAC 6 and HDAC 7 (Kao et al., 2000) have been cloned and identified (Grozinger et al., 1999). All share homology in the catalytic region. A fourth class I human HDAC was recently discovered, and was named HDAC 8, following the order of the appearance of the reports. HDAC9 and HDAC10 were also reported as being class II members. A third class of human histone deacetylases has been described belonging to the Sir2 family of proteins implicated in ageing mechanisms.

A variety of inhibitors for histone deacetylase have been identified. The proposed uses range widely, but primarily focus on cancer therapy. Saunders et al. (1999); Jung et al. (1997); Jung et al. (1999); Vigushin et al. (1999); Kim et al. (1999); Kitazomo et al. (2001); Vigusin et al. (2001); Hoffmann et al. (2001); Kramer et al. (2001); Massa et al. (2001); Komatsu et al. (2001); Han et al. (2001). They are the subject of an NIH sponsored Phase I clinical trial for solid tumors and non-Hodgkin's lymphoma and also have been shown to increase transcription of transgenes, thus constituting a possible adjunct to gene therapy. Yamano et al. (2000); Su et al. (2000).

Perhaps the most widely known is Trichostatin A, a hydroxamic acid-containing compound. It has been shown to induce hyperacetylation and cause reversion of ras transformed cells to normal morphology (Taunton et al., 1996) and induces immunosuppression in a mouse model (Takahashi et al., 1996). It is commercially available from BIOMOL Research Labs, Inc., Plymouth Meeting, Pa. and from Wako Pure Chemical Industries, Ltd. Also included in the present invention are trichostatins B and C, trapoxins A and B, chlamydocin, sodium butyrate, sodium phenylbutyrate, MS27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, m-carboxycinnamic acid bis-hydroxamide, and FK228. Various HDAC inhibitors are shown in Table 1.

The application is not limited to the listed HDAC inhibitors as, for example, Sternson et al. (2001) identified additional HDAC inhibitors using trichostatin A and trapoxin B as models. Additionally, the following references describe histone deacetylase inhibitors which may be selected for use in the current invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. 2002/103192; U.S. 2002/65282; U.S. 2002/61860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Furumai et al. (2002); Hinnebusch et al. (2002); Mai et al. (2002); Vigushin et al. (2002); Gottlicher et al. (2001); Jung (2001); Komatsu et al. (2001); Su et al. (2000).

TABLE 1

| Inhibitor | Compound Type | Chemical Make-Up | Organism |
|---|---|---|---|
| Trapoxin B | porphyrin derivative | C₃₃H₃₀N₄O₆ | H. ambiens |
| MS-27-275 | benzamide derivative | C₂₁H₂₀N₄O₃ | |
| Scriptaid | hydroxamic acid | C₁₈H₁₂N₂O₄ | |
| FR901228 | cyclopeptide | C₂₄H₃₆N₄O₆S₂ | C. violaceum (#968) |
| Depudecin | fungal metabolite | C₁₁H₁₆O₄ | A. brassiciola |
| Oxamflatin | aromatic sulfonamide | C₁₈H₁₄N₂O₄S₁ | |
| Pyroxamide (suberoyl-3-aminopyridineamide hydroxyamic acid) | hydroxamic acid | C₁₃H₂₀N₃O₃ | |
| 2-amino-8-oxo-9,10-epoxy-decanoyl (AEO) | ketone | C₁₀H₁₇NO₃ | |
| 3-(4-aroyl-1 H-pyrrol-2-yl)-N-hydroxy-2-propenamide | propenamide | C₁₄H₁₂N₂O₃ | |
| Suberoylanilide hydroxamic acid | hydroxamic acid | C₁₄H₂₀N₂O₃ | |
| m-Carboxycinnamic acid bis-hydroxamide | hydroxamic acid | C₁₀H₁₀N₂O₄ | |
| Apicidin¹ | cyclopeptide | C₂₉H₃₈N₅O₆ | Fusarium spp. |
| CHAP1 (trichostatin A + trapoxin B) | hydroxamic/porphyryin derivatives | | |

¹cyclo(N-O-methyl-L-tryptophanyl-L-isoleucinyl-D-pipecolinyl-L-2-amino-8-oxodecanoyl)

IV. Therapeutic Regimens

Among experts, there is an increasing tendency to treat AD, MS and ALS as expeditiously and aggressively as possible. In this scenario, one could use HDAC inhibitors to treat the first attack of demyelination, prior to an official diagnosis of MS, in order to try to arrest or ameliorate severity of progression to disability from early on. One may also treat formally diagnosed patients that have not received any other therapy. Alternatively, where another therapy has been applied and the attainable benefit achieved, the HDAC inhibitor may be used as an alternative treatment.

In another embodiment, the HDAC inhibitors of the present invention may be used in combination with other agents to improve or enhance the therapeutic effect of either. This process may involve administering both agents to the patient at the same time, either as a single composition or pharmacological formulation that includes both agents, or by administering two distinct compositions or formulations, wherein one composition includes the HDAC inhibitor and the other includes the second agent(s).

The HDAC therapy also may precede or follow the other agent treatment by intervals ranging from minutes to weeks. In embodiments where the other agent and HDAC inhibitor are administered separately, one may prefer that a significant period of time did not expire between the time of each delivery, such that the agent and HDAC inhibitor would still be able to exert an advantageously combined effect. In such instances, it is contemplated that one may administer both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other embodiments, it may be desirable to alternate the compositions so that the subject is not tolerized.

Various additional combinations may be employed, HDAC inhibitor therapy is “A” and the secondary agent is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

It is expected that the treatment cycles would be repeated as necessary.

Various drugs for the treatment of AD, MS and ALS are currently available as well as under study and regulatory consideration. For MS, drugs include steroids, such as ACTH, methylprednisolone, prednisolone; interferons such as interferons β1a (Avonex®, Rebif®) and β1b (Betaseron®), Copaxone® (known generically as glatiramer acetate), and Novantrone® (mitoxantrone). For ALS, drugs include gabapentin (Neurontin®), Myotrophin® (Insulin-like Growth Factor 1, IGF-1), brain-derived neurotrophic factor (BDNF), BFGF, Rilutek® (riluzole), SR57746A, metal chelators (e.g., D-penicillamine), creatine, cyclosporin, CoQ10, inhibitors of tubulin/filament assembly and various vitamins (e.g., C, E and B). For AD, the drugs generally fit into the broad categories of cholinesterase inhibitors, muscarinic agonists, anti-oxidants or anti-inflammatories. Galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol and neuromidal are just some of the drugs proposed as therapeutic agents for Alzheimer's disease.

V. Pharmaceutical Formulations and Routes of Administration

Pharmaceutical compositions of the present invention comprise an effective amount of an HDAC inhibitor and/or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one HDAC inhibitor or additional active ingredient will be known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularlly, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the compounds of the present invention are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

VI. Animal Models

A. Model for MS

Experimental autoimmune encephalomyelitis (EAE) is the most widely accepted animal model for multiple sclerosis. Thomas Rivers at the Rockefeller Institute in New York showed, in 1935, that brain tissue injections in monkeys induced EAE. Medications that currently modify the disease course in humans also modify the disease course in the animal model. The inventor has used the C57BL/6J mice (female mice 6-8 weeks old from Jackson Laboratories) for the experiments, which involve the administration of myelin oligodendrocyte glycoprotein (MOG), a myelin antigen, that induces autoimmunity against the brain tissue. EAE can also be induced in laboratory mice and rats using other myelin antigens.

B. Model for ALS

The SOD1 mutant animal model is widely accepted as the best model of human ALS. It has been particularly helpful in drug studies (Gurney, 1997). B6/SJL SOD1 mice that are transgenic for the G93A mutation (Stock No. 002726, B6SJL-TGN(SOD1-G93A)1Gur, Jackson Laboratories, Bar Harbor, Me.) and carry a high copy number of this mutant allele (often referred to as G1H) were used in these experiments. These hemizygous animals develop first signs of clinical disease at age 90-100, and reach end-stage disease by age 130-140 days. An SOD1 mutant rat model that develops motor neuron disease has also been generated (Nagai et al., 2001).

C. Model for AD

Mouse models with clinical features suggestive of AD have been generated. The amyloid beta (A4) precursor protein (APP) targeted mutation mice were generated by Dr. David Borchelt and can be purchased from The Jackson Laboratory (Bar Harbor, Me.). This mouse model develops decreased forelimb grip strength and locomotor activity. In addition, reactive astrocytosis can be demonstrated by histopathology by 14 weeks of age. The double transgenic APP (chimeric-mouse/human)-presenilin 1 (human), also generated by Dr. David Borchelt, can also be obtained from the Jackson Laboratory. The latter mice start accumulating amyloid deposits in the brain by nine months of age, similar to those found in human AD brains. These deposits increase dramatically by age 12 months. AD mouse models develop behavioral alterations that can be assessed using various tests, including the water maze, T maze, or contextual fear conditioning tests. Thus, a drug proposed to ameliorate AD in humans can be assessed and validated on the AD animal models. Other AD mice with various levels of expression of APPs have been generated, including animals that develop signs of disease or synaptic toxicity prior to plaque formation (Mucke et al., 2000). Models with the various mutations leading to AD-like pathology are reviewed in Price and Sisodia (1998).

VII. Screening Methods

In accordance with the present invention, there also are provided methods for screening drugs or drug combinations for efficacy in treating AD, MS and/or ALS. Primarily, these methods will rely upon the models described above, but they could easily be adapted to any other suitable assay system, both in vitro and in vivo.

In an exemplary assay, an HDAC inhibitor is provided to an experimental animal via an appropriate route. One or more symptoms of AD, MS or ALS are then assessed and compared to those seen in a similar animal not receiving the inhibitor, e.g., the same animal prior to receiving the inhibitor. Such symptoms include, but are not limited to:

ALS—focal or generalized motor weakness including progressive inability to walk or use limbs, spasticity, respiratory insufficiency, inability to swallow, choking, weight loss, muscle atrophy, muscle fasciculations, increased reflexes, and/or shortened life span

MS—dementia symptoms, decreased concentration, memory loss, blindness, decreased vision, decreased visual depth perception, abnormal eye movements, facial pain, abnormal facial movements, choking, muscular weakness, limb spasms/cramps, inability to walk due to weakness and incoordination, muscle atrophy, stiffness, impotence, intolerance to heat, focal or generalized pain, reflex sympathetic dystrophy, inability to perceive vibration or position changes, fatigue, tiredness, head titubation, tremors, or loss of balance

AD—decreased locomotor activity, decreased grip strength, inability to perform on water maze or T maze tests, impaired contextual fear conditioned responses.

A positive result might be interpreted as the diminution of a symptom, the delay, or prevention in appearance of a previously unseen symptom, or the delay or prevention of progression of an existing symptom.

The method may also comprise screening an HDAC inhibitor in combination with another agent. Thus, depending on whether one was more interested in examining the inhibitor, the other agent or the combination, the appropriate control would be an animal untreated with the inhibitor, the other agent, or both, respectively.

The assay may also comprise various other parameters, including timing of administration, varying the dose, assessing toxicity.

VIII. Identifying Subjects Having MS, ALS and AD

In various aspects of the invention, it will be desirable to identify subjects that have MS or ALS. The general approaches for diagnosis of these diseases are set out below. It also may be desirable to identify those individuals having increased risk for MS or ALS. At present, there are no truly prognostic tests. However, any of the following diagnostic procedures may be applied to individuals with few or no overt symptoms of MS or ALS and, in this way, provide early treatment that may prevent related neuropathologic damage and/or progression of the disease to a more clinically significant stage.

A. Multiple Sclerosis

Currently, there is no clear test that can definitely identify a person with MS. In addition, some symptoms of MS can be caused by other diseases. Thus, the diagnosis of MS must be made carefully. The basic “rule” for diagnosing MS relies on two criteria: (a) first, there must have been two attacks at least one month apart, an attack being defined as a sudden appearance of or worsening of an MS symptom or symptoms which lasts at least 24 hours; and (b) second, there must be more than one area of damage to central nervous system myelin, and the damage must have occurred at more than one point in time.

MRI (magnetic resonance imaging) currently is the preferred method of imaging the brain to detect the presence of plaques or scarring caused by MS. Often brains that appear to be normal on CT scans will show plaques with MRI. Still, the diagnosis of MS cannot be made solely on the basis of MRI as there are other diseases that cause lesions in the brain that look like those caused by MS. There also are lesions found in healthy individuals, particularly in older persons, which are not related to any ongoing disease process.

On the other hand, a normal MRI also does not rule out presence of MS. In fact, about 5% of patients who are confirmed to have MS on the basis of other criteria do not show any lesions in the brain on MRI. Rather, these individuals may have lesions in the spinal cord, or may have lesions which cannot be detected by MRI. Other symptoms will be evaluated during the clinical examination conducted by a physician to identify these subjects. Such examinations cover an extensive review of mental, emotional, and language functions, movement and coordination, vision, balance, and the functions of the five senses. Sex, birthplace, family history, and age of the person when symptoms first began are also taken into consideration.

Other tests that can be performed include evoked potentials, cerebrospinal fluid, and blood. Evoked potential tests are electrical diagnostic studies that can show if there is a slowing of messages in the various parts of the brain or spinal cord. They suggest scarring along nerve pathways that is not apparent on a neurologic exam. Cerebrospinal fluid may be tested for levels of certain immune system proteins and for the presence of oligoclonal bands. These bands indicate an immune response within the central nervous system. Oligoclonal bands are found in the spinal fluid of about 85-90% of people with MS. Since they are present in other diseases as well, oligoclonal bands alone cannot be relied on as positive proof of MS.

B. Amyotrophic Lateral Sclerosis

No single test provides a definitive diagnosis of ALS, although the presence of upper and lower motor neuron signs in a single limb is strongly suggestive. Rather, diagnosis of ALS is primarily based on symptoms the physician observes, and from a series of tests that help rule out other diseases. Because symptoms of ALS can be similar to those of other disorders, appropriate tests must be conducted to exclude the possibility of these other conditions. Electromyography (EMG) is a special recording technique that detects electrical activity in muscles. Certain EMG findings can support the diagnosis of ALS, and the electrophysiological criteria suggesting ALS have undergone several revisions over the past few years. Specific abnormalities in nerve conduction velocity (NCV) may suggest that the patient has a form of peripheral neuropathy (damage to peripheral nerves) or myopathy (muscle disease) rather than ALS. Magnetic resonance imaging (MRI) scans are often normal in patients with ALS, but can reveal evidence of other problems that may be causing the symptoms.

Based on these tests, the physician may order tests on blood and urine samples to eliminate other diseases. In some cases, if a physician suspects that the patient may have a myopathy rather than ALS, a muscle biopsy may be performed. Infectious diseases such as HIV, HTLV and Lyme disease also can cause ALS-like symptoms, as can multiple sclerosis, post-polio syndrome, multifocal motor neuropathy, and spinal muscular atrophy. Thus, they should be considered by physicians attempting to make a diagnosis.

C. Alzheimer's Disease

In various aspects of the invention, it will be desirable to identify subjects that have AD. The general approaches for diagnosis of these diseases are set out below. It also may be desirable to identify those individuals having increased risk for AD. At present, there are no truly prognostic tests. However, any of the following diagnostic procedures may be applied to individuals with few or no overt symptoms of AD and, in this way, provide early treatment that may prevent related neuropathologic damage and/or progression of the disease to a more clinically significant stage.

The diagnosis of both early (mild) cognitive impairment and AD are based primarily on clinical judgment. However, a variety of neuropsychological tests aid the clinician in reaching a diagnosis. Early detection of only memory deficits may be helpful in suggesting early signs of AD, since other dementias may present with memory deficits and other signs. Cognitive performance tests that assess early global cognitive dysfunction are useful, as well as measures of working memory, episodic memory, semantic memory, perceptual speed and visuospatial ability. These tests can be administered clinically, alone or in combination. Examples of cognitive tests according to cognitive domain are shown as examples, and include “Digits Backward” and “Symbol Digit” (Attention), “Word List Recall” and “Word List Recognition” (Memory), “Boston Naming” and “Category Fluency” (Language), “MMSE 1-10” (Orientation), and “Line Orientation” (Visuospatial). Thus, neuropsychological tests and education-adjusted ratings are assessed in combination with data on effort, education, occupation, and motor and sensory deficits. Since there are no consensus criteria to clinically diagnose mild cognitive impairment, various combinations of the above plus the clinical examination by an experienced neuropsychologist or neurologist are key to proper diagnosis. As the disease becomes more manifest (i.e. becomes a dementia rather than mild cognitive impairment), the clinician may use the criteria for dementia and AD set out by the joint working group of the National Institute of Neurologic and Communicative Disorders and Stroke/AD and Related Disorders Association (NINCDS/ADRDA). On occasion, a clinician may request a head computed tomography (CT) or a head magnetic resonance imaging (MRI) to assess degree of lobar atrophy, although this is not a requirement for the clinical diagnosis.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Efficacy of HDAC inhibitor treatment in reducing the clinical manifestations of experimental autoimmune encephalomyelitis (EAE) in mice. The inventor injected 6-8 week-old C57BL/6 female mice (Jackson Laboratories, Bar Harbor, Me.) subcutaneously with 150 μg myelin oligodendrocyte glycoprotein (MOG)₃₅₋₅₅ peptide (Quality Controlled Biochemicals, Hopkinton, Mass.) in PBS and CFA containing 0.4 mg of Mycobacterium tuberculosis (H37Ra, Difco, Detroit, Mich.), and i.p. on days 1 and 3 with 200 ng Pertussis (List Biological, Campbell, Calif.). TSA (7.5 mg/kg/dose/d i.p.) (Biomol, Plymouth Meeting, Pa. and Wako, Richmond, Va.) in PBS (9):DMSO (1) vehicle was begun on day 4. Ten mice were given vehicle and 9 were given TSA in a first experiment, wherease a second experiment utilized 15 per group.

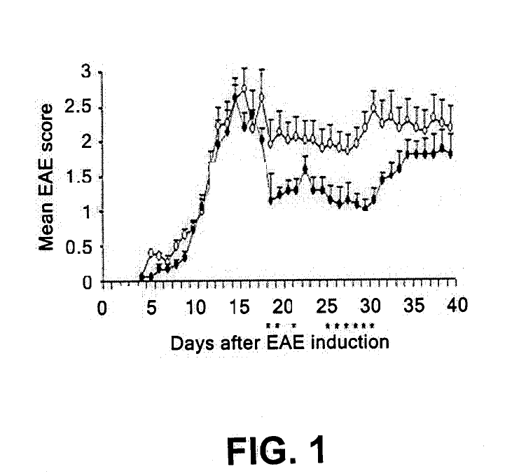

Treatment with TSA resulted in a decrease in EAE clinical disability that became apparent in the chronic (days 20-40) phase. FIG. 1. This latter phase of EAE is thought to better reflect the “neurodegenerative” component of EAE. The mean peak at remission and the mean percentage of days of severity, on data pooled from both separate experiments, was significantly decreased in TSA-treated mice.

TABLE 2
Clinical EAE

| | Vehicle | TSA |
|---|---|---|
| Incidence | 24/25 (96%) | 24/24 (100%) |
| Mortality | 2/25 (8%) | 2/24 (8.3%) |
| Days of Onset | 12.6 ± 0.5 | 13 ± 0.5 |
| Mean Peak Remission Phase | 2.5 ± 0.5 | 1.8 ± 0.1* |
| Mean % Days of Severity | 57.7 ± 5.3 | 33.9 ± 4.3** |

Summary of clinical findings of two separate exp. reveals that TSA-treated mice had a significant drop in the mean peak of the remission phase (*P = 0.0036) and in the mean percentage of days of severity (** P = 0.0011) by Fisher's PLSD. Two animals died at the time of the peak from the TSA group, and one animal died from the PBS group, but animals receiving TSA have clear improvement (i.e., sharp reduction) in signs of disability shortly after reaching the peak of disease.

The results demonstrate that the HDAC inhibitor Trichostatin A is effective in reducing EAE in mice when given intraperitoneally.

### Example 2

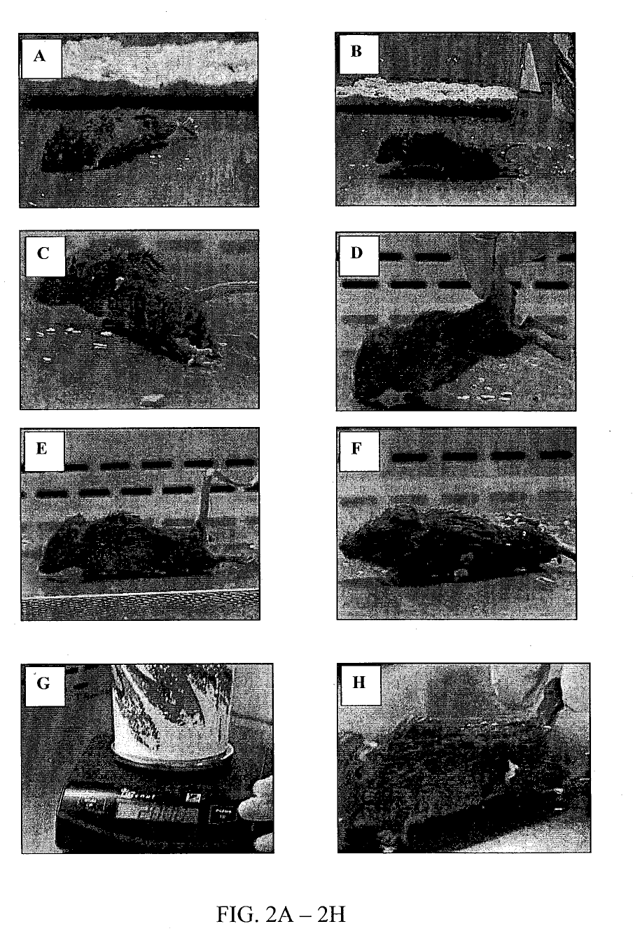

SOD1 G93A mutant ALS animals on no treatment on day 125. The animal in FIGS. 2A-2H looks emaciated, has significant muscle bulk loss, has stiffness (spasticity) predominantly involving the hind limbs (which are almost totally paralyzed) (FIGS. 2A-2C), has absence of extension reflex of the hind limbs when suspended by the tail (FIG. 2D), has spasticity and weakness of the tail (FIG. 2E), turns over on his side due to weakness (unable to maintain posture) (FIG. 2F), and weighs 20 g (FIG. 2G). Another ALS animal on no treatment exhibits similar characteristics, and, as shown, (FIG. 2H) had difficulty hyperextending the fore limbs in response to manipulation.

### Example 3

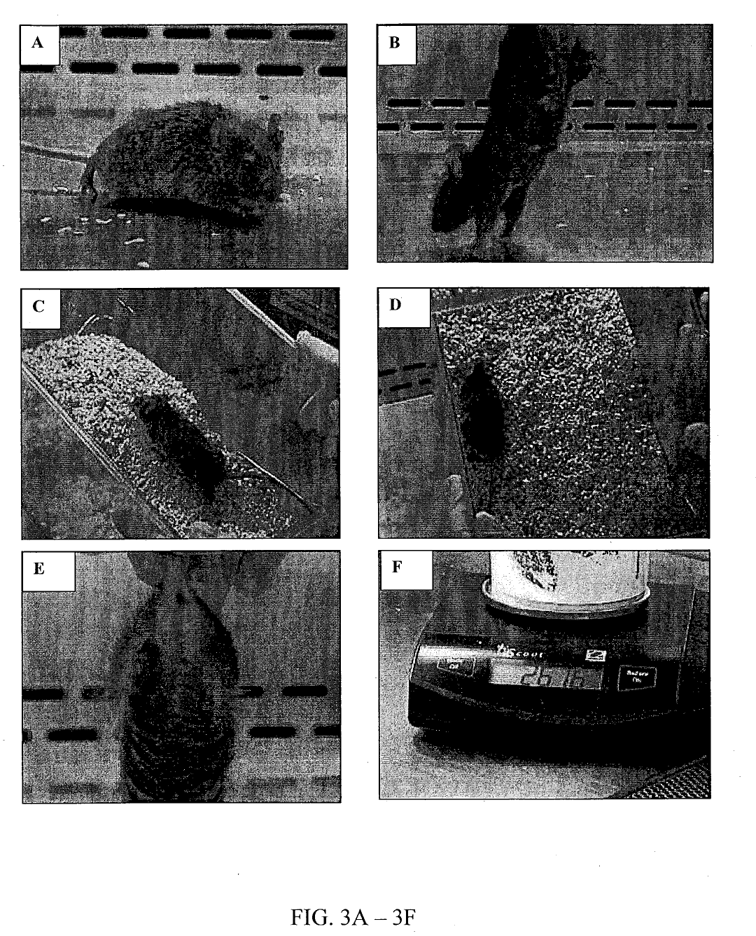

G93A mutant ALS mouse on oral sodium phenylbutyrate shown on day 125. Treatment consisted of 1 mg/ml in drinking water, equivalent to a calculated dose of 1 mg/mg of body weight/day (started on day 64 of life). Note that the treated animal has a normal appearance (FIG. 3A), has an appropriate limb extension reflex (FIG. 3B), walks upwards in the inclined cage (FIGS. 3C-D), only has tremors of hind limbs (an early sign of the disease, appreciated as a fuzzy image of the limbs in the picture) (FIG. 3E), and weighs 26.1 g (six grams more than the non-treated animals) (FIG. 3F). The tail is slightly weak (can not raise it fully upwards) (FIGS. 3A,3C, and 3D).

### Example 4

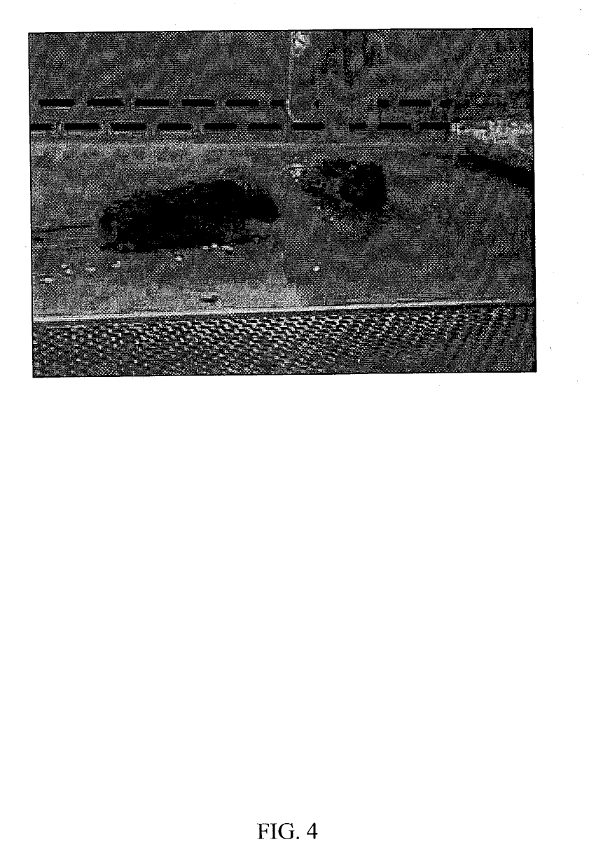

Comparison of SOD1 G93A ALS mice on day 125 of age. In FIG. 4, only the ALS animal on the left has been treated with oral sodium phenylbutyrate. Note the difference in body size, attributed to wasting syndrome and generalized muscle atrophy in the non-treated animal on the right.

### Example 5

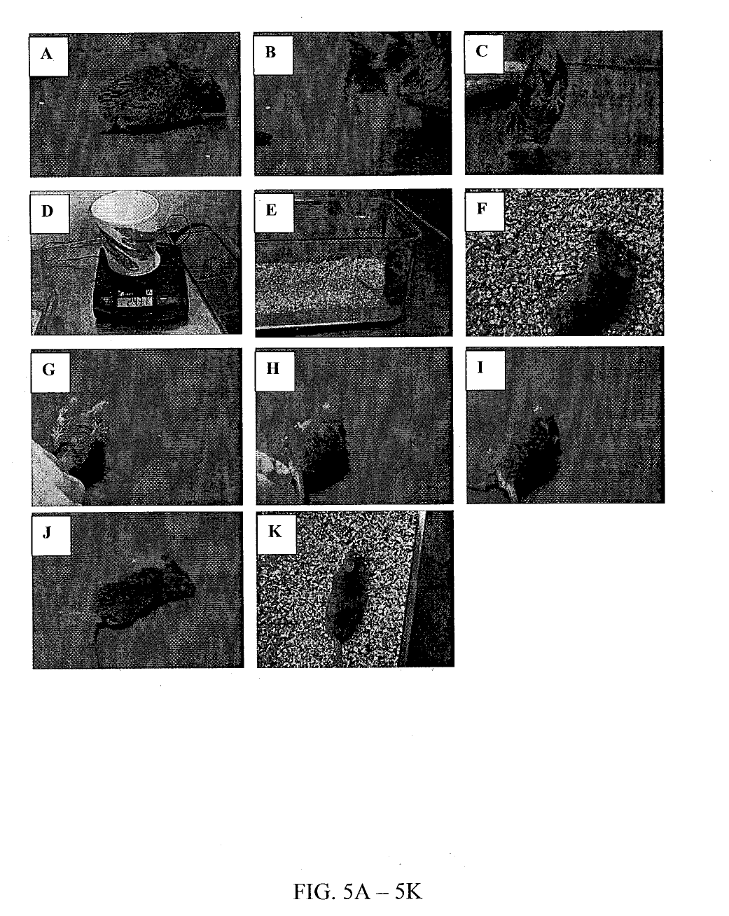

Phenylbutyrate-treated SOD1-mutant ALS animal at day 138. The same phenylbutyrate-treated animal that was shown previously at day 125 (FIG. 3), now shown (FIG. 5A) on day 138 of life (non-treated littermate controls in this study reached the end-stage at days 127 and 136). The animal still has resting tremor (FIG. 5B), is less agile and has a weak extension reflex when raised from the tail (FIG. 5C). In addition, its weight has dropped from 26 to 24.3 g (FIG. 5D). However, the animal is still able to ambulate, freely move all limbs, stand on its hind limbs by itself (FIG. 5E), raise its head (FIG. 5F), and is able to consistently regain posture when laid on its back (albeit slowly) (FIGS. 5G-4J). Its tail is weak, but not spastic. In the 45 degrees-inclined cage, the animal is able to slowly walk upwards and does not slide (FIG. 5K).

### Example 6

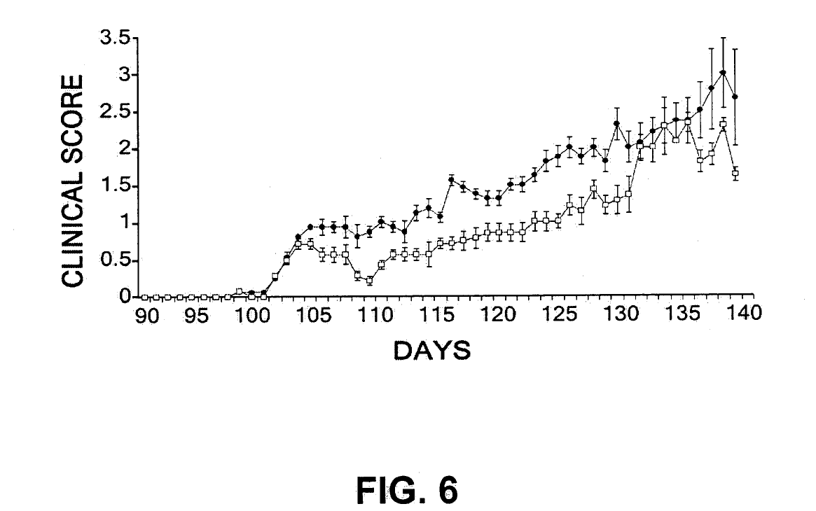

HDAC inhibitors ameliorate disability in ALS mice. Treatment of ALS SOD1G93A mice with oral sodium phenylbutyrate (SPB) (n=7) at a dose of 1 mg SPB/ml of drinking water, started on day 64 of age, resulted in a significant drop in disability scores, as compared to non-treated mice (n=8). FIG. 6.

### Example 7

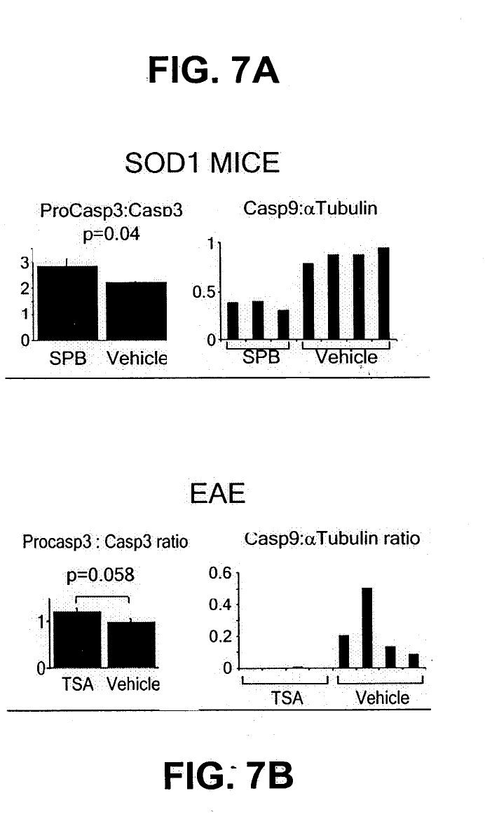

Mechanisms of protection by HDAC inhibitors. (1) Decreased caspase activation leading to enhanced neuronal survival. By western analysis, the inventor showed that oral sodium phenylbutyrate (SPB) treatment of ALS (SOD1 mutant) mice and intraperitoneal TSA treatment of MS (EAE) mice both result in decreased activation of caspases 3 and 9 in CNS tissue (FIGS. 7A-B). Caspase 3 is known to be activated in rat EAE spinal cords (Ahmed et al., 2002), and correlates with retinal ganglion cell loss and optic neuritis-related disability (Meyer et al., 2001). Neuronal overexpression of the anti-caspase factor Bcl-2 ameliorates EAE (Offen et al., 2000). Caspase 3 also has the ability to modulate Bcl2 and activate upstream caspases triggered by Fas (Woo et al., 1999), an apoptotic pathway implicated in EAE (Sabelko et al., 1997). Finally, CNS activation of caspases has been implicated in the progression of Alzheimer's disease (AD) (Pompl et al., 2003). Thus, the inventor proposes that blockade of neuronal caspase activation is a mechanism by which HDAC inhibitors ameliorate neuronal death in these disorders (EAE, ALS and AD).

(2) Modulation of genes involved in neuroprotection and immune regulation by TSA. a) Microarrays detect protective genes regulated by TSA in EAE mice spinal cords. The inventor pooled RNA from tissues of three animals in each group (TSA treated and vehicle-treated) for DNA microarray analysis. As shown (Table 3), intraperitoneal TSA led to the enhanced expression of mRNAs for the antioxidant glutathione peroxidase (Gpx1) and the neuroprotective insulin-like growth factor 2 (Igf2). These two genes were also upregulated by SPB in spinal cords of ALS mice. Intraperitoneal TSA also led to decreased expression in EAE spinal cords of the chemokine receptor CCR6, immunoglobulin V(H)II, the vascular homeostasis-related phospholipase A2, and upregulation of the immunomodulatory factor interferon-α2 (Ifna2), the latter with known beneficial effects in MS patients.

TABLE 3
Genes Altered by I.P. TSA in EAE Mice Spinal Cords

| Probe Set | Gene Description | Fold Change |
|---|---|---|
| 97680 | Murinoglobulin (Mug) | −7.2 |
| 99899 | CCR6 | −7.3 |
| 97540 | Histocompatibility 2 D region locus 1 | 7.4 |
| 101676 | Glutathione peroxidase (Gpx1) | 6.9 |
| 104364 | Mapkapk5 | −6.5 |
| 99326 | Phospholipase A2 (Pla2) | −6.4 |
| 92782 | Thymopoietin (Tmpo) | −6.4 |
| 97970 | Transferrin-like p97 | −5.7 |
| 94717 | IFN-alpha-2 (1fna2) | 5.6 |
| 92833 | Histidine ammonia lyase (Ha) | 5.3 |
| 92500 | Ten-m3 | −5.4 |
| 98623 | Insulin-like growth factor 2 (1gf2) | 5.2 |
| 94521 | Cdk inhibitor p19 | −5.3 |
| 93378 | Hox-3.1 | −5.3 |
| 100362 | Ig V(H)II | −4.9 |
| 93275 | SH3 domain protein 2B | 4.5 |

b) Microarrays detect protective genes regulated by TSA in EAE mice spleens. The inventor had previously shown that HDAC mRNAs are elevated in immune cells after stimulation with mitogens or α-CD3 Ab (Dangond et al., 1998) and that HDAC inhibitors block proliferation (Dangond and Gullans, 1998). HDAC inhibition also downregulates pro-inflammatory molecules, including γ-IFN (Dangond and Gullans, 1998), IL-12 (Saemann et al., 2000), IL-12 receptor (Saemann et al., 2000), B7-1 (Bohmig et al., 1997) and TNF-α (Nancey et al., 2002). Using microarrays, the inventors have been able to show that TSA treatment of EAE mice leads to downregulation of numerous immunoglobulin mRNAs in spleen tissue (Table 4). A known histone deacetylase-binding chaperone, 14-3-3 sigma, is also elevated in spleens.

TABLE 4
Genes Altered by I.P. TSA in EAE Mice Spleens

| Probe Set | Gene Description | Fold Change |
|---|---|---|
| 99671 | Adipsin (Adn) | 9.0 |
| 101115 | Lactotransferrin (Ltf) | −8.2 |
| 101752 | IgG variable | −8.2 |
| 96719 | Parvalbumin (Pva) | 6.3 |
| 102149 | Interferon alpha 7 (Ifna7) | 5.8 |
| 101870 | γ-1 Ig Constant | −5.0 |
| 101616 | IgG rearranged κ | −4.5 |
| 96704 | 14-3-3 sigma | 3.9 |
| 97570 | Igκ-V22 | −4.4 |
| 99837 | Galanin receptor 1 (Galr1) | −4.2 |
| 101826 | V kappa and J kappa coding joint | −4.2 |
| 96970 | IgH variable | −3.9 |
| 102843 | IgH gene DJC | −3.6 |
| 99850 | IgE H constant | −3.1 |
| 103830 | Snail homolog | 3.0 |
| 99420 | IgA VDJH | −2.8 |
| 100060 | Kallikrein (Klk) | 2.8 |

Microarrays detect protective genes co-regulated by TSA in EAE mice spinal cords and spleens. The inventor was able to show that intraperitoneal TSA treatment of EAE mice led to gene expression changes that were shared between tissues as diverse as spinal cord and spleen (Table 5). Of particular interest, the neuronal trait encoding neurofilament heavy (Nefh) gene was upregulated in these tissues by TSA. The growth differentiation factor 9 (Gdf9) was upregulated, and several genes of the Wnt signaling pathway, such as Axin, disheveled 2 and Frat1 were dysregulated by TSA. Several immunoglobulins were downregulated by this HDAC inhibitor. Genes encoding proteins that participate in HDAC- or HDAC complex-binding, such as Dnmt1 and ROX (Mnt) were downregulated by TSA in both tissues.

TABLE 5

| | | | |
|---|---|---|---|
| | | Fold Δ | Fold Δ |
| Probe Set | Gene Description | CNS | Spleen |

CYTOSKELETON

| | | | |
|---|---|---|---|
| M35131 | Neurofilament heavy (Nefh) | 2.3 | 2.2 |

INFLAMMATION

| | | | |
|---|---|---|---|
| X16678 | Immunoglobulin kappa chain (1gk-V20) | −3.3 | −2.3 |
| U62386 | Immunoglobulin H and L variable | −2.1 | −2.5 |

REDOX, STRESS OR MITOCHONDRIAL

| | | | |
|---|---|---|---|
| AV108173 | Metaxin homolog | −11.6 | −4 |
| X03920 | Glutathione peroxidase (Gpx1) | 3 | 2.3 |

SIGNAL TRANSDUCTION

| | | | |
|---|---|---|---|
| U58974 | Rearranged in lymphoma (Frat1) | 4.5 | 5.4 |
| U85714 | Phospholipase C-beta-1b (Picb1) | 3.4 | 3.7 |
| AF009011 | Axin | 4.9 | 2.1 |
| U24160 | Dishevelled 2 (Dvl2) | −2.1 | −3.6 |

TRANSCRIPTION AND EPIGENETIC

| | | | |
|---|---|---|---|
| U70017 | Cyclin D-interacting Dmp1 (Dmtf1) | 5.4 | 5.2 |
| AF036008 | DNA methyltransferase (Dnmt1) | −8.8 | −3.2 |
| M95604 | Snail homolog 1 (Snail1) | 5.3 | 2.9 |
| AB010557 | Paired box gene 4 (Pax4) | −12.5 | −2.4 |
| X55781 | Paired box gene 2 (Pax2) | 7.1 | 2.1 |
| Y07609 | Max binding protein ROX (Mnt) | −2.2 | −3.5 |
| U77967 | Neuronal PAS domain 1 (Npas1) | 2 | −3.5 |

VASCULAR HOMEOSTASIS

| | | | |
|---|---|---|---|
| D10849 | Thromboxane A2 receptor (Tbxa2r) | −5.2 | −4.9 |

GROWTH

| | | | |
|---|---|---|---|
| L06444 | Growth differentiation factor (Gdf9) | 3.5 | 5.4 |

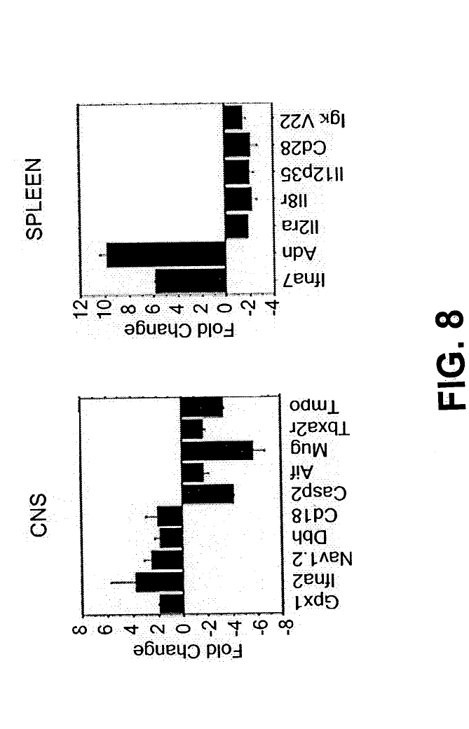

d) Quantitative RT-PCR (QRT-PCR) on CNS and spleen tissues from TSA-treated EAE mice detects numerous genes modulated by this HDAC inhibitor. These include genes previously shown by microarrays to be modulated by TSA, such as Gpx1 and Ifna2 (both elevated in the CNS by TSA), and Mug, thromboxane A2 receptor (Tbxa2r) and Tmpo (all three downregulated in the CNS by TSA). QRT-PCR unveiled new CNS effects of TSA (FIG. 8), including upregulation of the neuronal trait genes sodium channel Nav1.2 and dopamine β hydroxylase (Dbh), and downregulation of the pro-apoptotic factors caspase 2 (casp2) and apoptosis inducing factor (Aif). In spleen tissues, QRT-PCR confirmed elevation of Ifna7 and Adipsin and downregulation of Igκ V22 mRNAs, and unveiled downregulation by TSA of multiple inflammatory factors, such as IL-8 receptor, IL-2 receptor, IL-12, and the costimulatory molecule CD28, from pathways implicated in MS (Glabinsk and Ransohoff, 2001; Dangond, 2002).

HDAC inhibitors in vitro have been shown to upregulate TGF-beta receptor (Park et al., 2002), B7-2 (Bohmig et al. 1997), IL-4, IL-10 (Saemann et al, 2000), IL-6 (Wang et al., 1999) and β-IFN (Shestakova et al., 2001), the latter used currently for MS treatment. The inventors have demonstrated IFN-α CNS elevation by TSA which may play an important role in EAE amelioration, since IFN-α also benefits MS patients (Durelli et al., 1994). Remarkably, clinical improvement of EAE was also associated with a decrease by TSA of numerous immunoglobulin mRNAs in spleen and CNS tissues. This widespread anti-inflammatory action of TSA may translate into less neuronal exposure to oxidant stress- or cellular-mediated cell death signals.

Summary of HDAC inhibition-associated neuroprotection. Clearly, there are widespread effects triggered by inhibition of HDAC enzymes. The inventor has found that treatment of EAE with intraperitoneal TSA and of ALS mice with oral sodium phenylbutyrate led to upregulation, in the CNS of both animal models, of the neurotrophic insulin-like growth factor 2 (Igf2) and the anti-oxidant glutathione peroxidase 1 (Gpx1) genes. Oxidant stress leads to neuronal death (Ratan et al., 1994). Gpx1 is a key enzyme in reducing H₂O₂ (Jain et al., 1991) and detoxifying peroxynitrite (Sies et al., 1997). Gpx1 reduces blood brain barrier (BBB) permeability to serum factors in EAE (Guy et al., 1989). Other peroxynitrite scavengers, such as uric acid, also ameliorate EAE by reducing BBB permeability (Hooper et al., 1998). Thus, neurons from TSA-treated EAE mice may survive free radical attack during inflammation due to enhanced detoxification mechanisms and decreased BBB breakdown, via GPx1 upregulation.

TSA decreases the CNS levels of the caspase-independent Aif (Cregan et al., 2002) mRNA, as well as caspase 2 mRNA, but other mechanisms may enhance the ability of TSA to achieve neuroprotection as well. Derepression of neuronal traits in progenitor cells by HDAC inhibition is an attractive recruiting mechanism, since it has been shown that ventricular zone progenitors gradually drop their expression of transcription factor REST mRNA as they migrate to become neurons (Schoenherr and Anderson, 1995; Chong et al., 1995). TSA-induced expression of the known HDAC/REST-repressed gene Nav1.2 (Schoenherr et al., 1996), and of the neuronal gene Dbh, reported to be highly expressed in non-neuronal cells that lack REST expression (Atouf et al., 1997), suggests maintenance of a mature architecture by neurons or acquisition of the neural phenotype by non-neuronal differentiating cells.

It is clear from this invention that neuroprotection by HDAC inhibitors occurs at multiple levels by affecting the complex balance of transcriptional regulation, potently modulating the immune system, promoting anti-oxidant and growth responses, counteracting caspase-dependent and independent pro-apoptotic signals, and possibly derepressing neuronal integrity traits in vivo. The present invention opens a new field of possibilities for combating neurodegenerative disorders characterized by oxidant stress, apoptosis and inflammation, such as MS, ALS and AD.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.

U.S. Provisional Serial No. 60/368,228

U.S. Provisional Serial No. 60/404,664

U.S. Pub No. 2002/103192

U.S. Pub No. 2002/65282

U.S. Pub No. 2002/61860

AU 9,013,101

AU 9,013,201

AU 9,013,401

AU 6,794,700

EP 1,233,958

EP 1,208,086

EP 1,174,438

EP 1,173,562

EP 1,170,008

EP 1,123,111

JP 2001/348340

WO 02/51842

WO 02/50285

WO 02/46144

WO 02/46129

WO 02/30879

WO 02/26703

WO 02/26696

WO 01/70675

WO 01/42437

WO 01/38322

WO 01/18045

WO 01/14581

Ahmed et al., Am. J. Pathol., 161:1577-1586, 2001.

Atouf et al., J. Biol. Chem., 272:1929-1934, 1997.

Bohmig et al., Immunology, 92:234-243, 1997.

Chang et al., Proc. Natl. Acad. Sci. USA, 98(17):9808-9813, 2001.

Chong et al., Cell, 80:949-957, 1995.

Cregan et al., J. Cell Biol., 158:507-517, 2002.

Dangond and Gullans, Biochem. Biophys. Res. Commun., 247:833-837, 1998.

Dangond, In: Disorders of Myelin in the Central and Peripheral Nervous Systems, Dangond (Ed.), Butterworth Heinemann, Boston, 1:103-138, 2002.

Dangond et al., Biochem. Biophys. Res. Commun., 242:648-652, 1998.

Durelli et al., Neurology, 44:406-413, 1994.

Emiliani et al., Proc. Natl. Acad. Sci. USA, 95:2795-2800, 1998.

Furumai et al., Cancer Res., 62(17):4916-4921, 2002.

Glabinsk and Ransohoff, Curr. Opin. Investig. Drugs, 2:1712-1719, 2001.

Gottlicher et al., EMBO J, 20(24):6969-6978, 2001. −Grozinger et al, Proc. Natl. Acad. Sci. USA, 96:4868-4873, 1999.

Gurney, J. Neurol. Sci., 152(1):S67-73, 1997.

Guy et al., Arch. Ophthalmol., 107:1359-63, 1989.

Han et al., Cancer Research, 60:6068-6074, 2000.

Hinnebusch et al, J. Nutr., 132(5):1012-1017, 2002.

Hoffmann et al, Bioconjugate Chem., 12:51-55, 2001.

Hooper et al, Proc. Natl. Acad. Sci. USA, 95:675-680, 1998.

Issazadeh et al., J. Immunol. 161:1104-12, 1998.

Jain et al., Proc. Natl. Acad. Sci. USA, 88:913-917, 1991.

Jung et al., J. Med. Chem., 42:4669-4679, 1999.

Jung et al., Med. Chem. Lett., 7:1655-1658, 1997.

Jung, Curr. Med. Chem., 8(12):1505-1511, 2001.

Kao et al., Genes Dev., 14(1):55-66, 2000.

Kim et al., Oncogene, 18:2461-2470, 1999.

Kitazomo et al., J. Clinical Endocrinology Metabolism, 86(7):3430-3435.

Komastsu et al., Cancer Research, 61:4459-4466, 2001

Kramer et al., Trends Endocrin. Metabol., 12(7):294-300, 2001.

Mai et al., J. Med. Chem., 45(9):1778-1784, 2002.

Massa et al, J. Med. Chem., 44:2069-2072, 2001.

Meyer et al., J. Neurosci., 21:6214-6220, 2001.

Mucke et al, J. Neuroscience 20: 4050-4058(2000).

Nagai et al., J. Neurosci. 21:9246-54, 2001.

Nancey et al., Dig. Dis. Sci., 47:921-928, 2002.

Offen et al., J. Mol. Neurosci., 15:167-176, 2000.

Park et al., J. Biol. Chem., 277:5168-5174, 2002.

Pompl et al., Arch. Neurol., 60:369-376, 2003.

Price and Sisodia, Ann. Rev. Neurosci., 21:479-505, 1998.

Ratan et al., J. Neurochem., 62:376-379, 1994.

Remington's Pharmaceutical Sciences, 18th Ed. Mack Printing Company, pp. 1289-1329, 1990.

Sabelko et al., J. Immunol., 159:3096-3099, 1997.

Saemann et al., Faseb J, 14:2380-2382, 2000.

Saunders et al., Cancer Res., 59:399-409, 1999.

Schoenherr and Anderson, Science, 267:1360-1363, 1995.

Schoenherr et al., Proc. Natl. Acad. Sci. USA, 93:9881-9886, 1996.

Shestakova et al., J. Virol., 75:3444-3452, 2001.

Sies et al., J. Biol. Chem., 272:27812-27817, 1997.

Steffan et al., Nature, 413:739-43, 2001.

Sternson et al., Org. Lett., 3(26):4239-42, 2001.

Su et al., Cancer Res., 60:3137-3142, 2000.

Takahashi et al., Antibiotics, 49:453, 1996.

Taunton et al., Science, 272:371-411, 1996.

Vigushin et al., Clinical Cancer Research, 7:971-976, 2001.

Vigushin et al., Anticancer Drugs, 13(1):1-13, 2002.

Wang et al., Br. J. Cancer, 80:1617-1622, 1999.

Woo et al., J. Immunol., 163:4909-4916, 1999.

Workman and Kingston, Annu. Rev. Biochem., 67:545-579,1998.

Yamano et al., Mol. Ther., 1(6):574-80 2000.

Yang et al., Proc. Natl. Acad. Sci. USA, 93:12845-12850, 1996.

Yang et al., J. Biol. Chem., 272:28001-28007, 1997.

What is claimed is:

1. A method for treating or preventing amyotrophic lateral sclerosis (ALS) in a human subject comprising administering to said subject a therapeutic amount of a histone deacetylase (HDAC) inhibitor.

2. The method of claim 1, wherein said HDAC inhibitor is selected from the group consisting of trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228 and m-carboxycinnamic acid bis-hydroxamide.

3. The method of claim 1, wherein treating comprises reducing one or more symptoms of ALS.

4. The method of claim 3, wherein said symptoms comprise focal or generalized motor weakness including progressive inability to walk or use limbs, spasticity, respiratory insufficiency, inability to swallow, choking, weight loss, muscle atrophy, muscle fasciculations, increased reflexes, progressive inability to perform activities of daily living, and/or shortened life span.

5. The method of claim 1, wherein treating comprises inhibiting the progression of ALS.

6. The method of claim 1, wherein preventing comprises identifying a subject at risk of ALS.

7. The method of claim 1, wherein said HDAC inhibitor is administered orally, intraperitoneally, intrathecally, intravenously, intranasally, intraparenchymally, subcutaneously, intramuscularly, intravenously, dermally, and intrarectally.

8. The method of claim 1, further comprising administering to said subject a second agent.

9. The method of claim 8, wherein said second agent is a second HDAC inhibitor.

10. The method of claim 8, wherein said second agent is selected from the group consisting of Riluzole.

11. The method of claim 8, wherein said second agent is provided before said HDAC inhibitor.

12. The method of claim 8, wherein said second agent is provided after said HDAC inhibitor.

13. The method of claim 8, wherein said second agent is provided at the same time as said HDAC inhibitor.

14. The method of claim 9, wherein said second HDAC inhibitor is provided in a different route than the first HDAC inhibitor.

15. The method of claim 8, wherein said second agent is administered orally, intraperitoneally, intrathecally, intravenously, intranasally, intraparenchymally, subcutaneously, intramuscularly, intravenously, dermally, and intrarectally.

16. A method for treating or preventing multiple sclerosis (MS) in a human subject comprising administering to said subject a therapeutic amount of a histone deacetylase (HDAC) inhibitor.

17. The method of claim 16, wherein said HDAC inhibitor is selected from the group consisting of trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228 and m-carboxycinnamic acid bis-hydroxamide.

18. The method of claim 16, wherein treating comprises reducing one or more symptoms of MS.

19. The method of claim 18, wherein said symptoms comprise dementia symptoms, decreased concentration, memory loss, inappropriate social affect, bipolar disorder symptoms, social disinhibition, decreased visuospatial abilities, blindness, decreased vision, decreased visual depth perception, decreased gaze fixation, ocular pain, abnormal eye movements, facial pain, abnormal facial movements, tinnitus, hoarse speech, choking, urinary incontinence, urgency, hesitancy, or retention, fecal incontinence, constipation, or obstipation, muscular weakness, limb spasms/cramps, inability to walk or grab objects due to weakness and incoordination, muscle atrophy, stiffness, impotence, loss of libido, vaginal pain or numbness sensation, pelvic spasms, anorgasmia, tingling, numbness, abnormal sensory perception, intolerance to heat, focal or generalized pain, sciatica pain, reflex sympathetic dystrophy, inability to perceive vibration or position changes, electric shock-like sensation going down the spine or limbs following flexion of the neck, fatigue, tiredness, head titubation, tremors, loss of balance, slurred speech, vertigo, or recurrent clinical deterioration.

20. The method of claim 16, wherein treating comprising inhibiting the progression of MS.

21. The method of claim 16, wherein preventing comprises identifying at subject at risk of MS.

22. The method of claim 16, wherein said HDAC inhibitor is administered orally, intraperitoneally, intrathecally, intravenously, intranasally, intraparenchymally, subcutaneously, intramuscularly, intravenously, dermally, and intrarectally.

23. The method of claim 16, further comprising administering to said subject a second agent.

24. The method of claim 23, wherein said second agent is a second HDAC inhibitor.

25. The method of claim 23, wherein said second agent is selected from the group consisting of methylprednisolone, prednisolone, interferon-β1a, interferon-β1b, glatiramer acetate, and mitoxantrone.

26. The method of claim 23, wherein the second agent is provided before said HDAC inhibitor.

27. The method of claim 23, wherein the second agent is provided after said HDAC inhibitor.

28. The method of claim 23, wherein the second agent is provided at the same time as said HDAC inhibitor.

29. The method of claim 24, wherein said second HDAC inhibitor is provided in a different route than the first HDAC inhibitor.

30. The method of claim 23, wherein said second agent is administered orally, intraperitoneally, intrathecally, intravenously, intranasally, intraparenchymally, subcutaneously, intramuscularly, intravenously, dermally, and intrarectally.

31. A method of screening a histone deacetylase (HDAC) inhibitor for use in treating or preventing amyotrophic lateral sclerosis (ALS) or multiple sclerosis (MS) comprising:
(a) providing a suitable animal model for ALS or MS;
(b) administering at least a first HDAC inhibitor to said animal; and
(c) assessing one or more symptoms of ALS or MS on said animal,
wherein an improvement in said one or more symptoms, as compared to a comparable animal not treated with said HDAC inhibitor, indicates that said HDAC inhibitor is useful in treating or preventing ALS or MS.

32. The method of claim 31, wherein said method screens for ALS therapy and said animal model is the SOD1 G93A mutant mouse.

33. The method of claim 31, wherein said method screens for MS therapy and said animal model is experimental autoimmune encephalomyelitis (EAE).

34. The method of claim 31, wherein said symptoms comprise weakness, paralysis, ataxia, blindness, tremors, spasticity, incontinence, and/or abnormal behavior.

35. The method of claim 31, further comprising administering to said animal a second agent.

36. The method of claim 35, wherein said second agent is a second HDAC inhibitor or is selected from the group consisting of methylprednisolone, prednisolone, interferon-β1a, interferon-β1b, glatiramer acetate, and mitoxantrone for MS and Riluzole for ALS.

37. The method of claim 35, further comprising comparing said one or more symptoms to a comparable animal when treated with said HDAC inhibitor or said second agent alone.

38. The method of claim 35, wherein the second agent is provided before said HDAC inhibitor.

39. The method of claim 35, wherein the second agent is provided after said HDAC inhibitor.

40. The method of claim 35, wherein the second agent is provided at the same time as said HDAC inhibitor.

41. A pharmaceutical composition comprising an HDAC inhibitor and a drug useful for treating amyotrophic lateral sclerosis and/or multiple sclerosis.

42. The composition of claim 41, where said HDAC inhibitor is selected from the group consisting of trichostatins A, B and C, trapoxins A and B, chlamydocin, sodium butyrate, sodium phenylbutyrate, MS-27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228 and m-carboxycinnamic acid bis-hydroxamide.
